(19) United States
(12) Patent Application Publication
(10) Pub. No.: US 2024/0112335 A1
(43) Pub. Date: Apr. 4, 2024

(54) COMPUTER-IMPLEMENTED METHOD FOR A POST-ACQUISITION CHECK OF AN X-RAY IMAGE DATASET

(71) Applicant: Siemens Healthcare GmbH, Erlangen (DE)

(72) Inventors: Andreas FIESELMANN, Erlangen (DE); Christian HUEMMER, Lichtenfels (DE); Ramyar BINIAZAN, Nuernberg (DE)

(73) Assignee: Siemens Healthcare GmbH, Erlangen (DE)

(21) Appl. No.: 18/476,643

(22) Filed: Sep. 28, 2023

(30) Foreign Application Priority Data
Sep. 29, 2022 (EP) 22198712.6

(51) Int. Cl. G06T 7/00; G16H 30/20

(52) U.S. Cl. CPC G06T 7/0012; G16H 30/20; G06T 2207/10116; G06T 2207/20081

(57) ABSTRACT

A computer-implemented method comprises: receiving input data, wherein the input data includes the X-ray image dataset, which includes an X-ray image and first metadata; applying a trained function to the input data to generate output data, wherein the output data includes second metadata, and wherein the first metadata and the second metadata are compared; and providing the output data, wherein the first metadata are confirmed in case the first metadata and the second metadata agree, or the first metadata are suggested to be corrected with the second metadata in case the first metadata and the second metadata do not agree.

### CROSS-REFERENCE TO RELATED APPLICATION(S)

The present application claims priority under 35 U.S.C. § 119 to European Patent Application No. 22198712.6, filed Sep. 29, 2022, the entire contents of which are incorporated herein by reference.

### FIELD

One or more example embodiments of the present invention relate to a computer-implemented method for a post-acquisition check of an X-ray image dataset.

### BACKGROUND

An X-ray imaging system, e.g. a radiography system or a fluoroscopy system, comprises an X-ray source and an X-ray detector. The examination object, in particular a patient, is arranged between the X-ray source and the X-ray detector so that an X-ray image of an examination region can be acquired.

In clinical routine, the human operator of a medical imaging device (e.g. X-ray system) must select the image acquisition protocol used for a specific organ to be acquired. It may happen that the operator accidentally or systematically selects an unsuitable protocol (e.g. a chest protocol for a hand to be acquired).

As an example, in an statistical analysis of a PACS dataset it has been found that the body part examined was incorrectly labeled in in 1% of cases and the selected clinical protocols (e.g., chest for hand images) has been incorrectly selected in 0.2% of cases. Wrongly chosen protocols lead to the following (clinical and technical) problems: Inadequately selected image acquisition protocols can lead to insufficient image quality, or more than the necessary dose applied to an organ; inadequately selected image acquisition protocols can lead to incorrectly labeled images (e.g. incorrect body part examined) and limit the interoperability of those images in the clinical workflow or could even lead to wrong diagnosis.

### SUMMARY

In current clinical routine there is no dedicated check that the image acquisition protocol has been correctly selected. The operator of the imaging system or the radiologist interpreting the X-ray image may recognize by looking at the image and the meta information that the protocol was incorrectly selected.

It is an object of one or more example embodiments of the present invention to provide a computer-implemented method for a post-acquisition check of an X-ray image dataset, a computer-implemented method for providing a trained function, a checking system, a computer program product, a computer-readable medium, a training system, and an X-ray system, which allows a correction of the metadata before review of the X-ray image or at the time of the review of the X-ray image.

At least the above-mentioned object of one or more example embodiments of the present invention is solved by a computer-implemented method for a post-acquisition check of an X-ray image dataset according to claim 1, a computer- implemented method for providing a trained function according to claim 10, a checking system according to claim 11, a computer program product according to claim 12, a computer-readable medium according to claim 13, a training system according to claim 14, and an X-ray system according to claim 15.

In the following, the solution according to embodiments of the present invention is described with respect to the claimed systems as well as with respect to the claimed methods. Features, advantages or alternative embodiments herein can be assigned to the other claimed objects and vice versa. In other words, claims for the systems can be improved with features described or claimed in the context of the methods. In this case, the functional features of the method are embodied by objective units of the system.

Furthermore, in the following, the solution according to embodiments of the present invention is described with respect to methods and systems for a post-acquisition check of an X-ray image dataset as well as with respect to methods and systems for the training of the trained function. Features, advantages or alternative embodiments herein can be assigned to the other claimed objects and vice versa. In other words, claims for methods and systems for training of the trained function can be improved with features described or claimed in context of the methods and systems for a post-acquisition check of an X-ray image dataset, and vice versa.

In particular, the trained function of the methods and systems for a post-acquisition check of an X-ray image dataset can be adapted by the methods and systems for training of the trained function. Furthermore, the input data can comprise advantageous features and embodiments of the training input data, and vice versa. Furthermore, the output data can comprise advantageous features and embodiments of the output training data, and vice versa.

At least one embodiment of the present invention relates to a computer-implemented method for a post-acquisition check of an X-ray image dataset, comprising: Receiving input data, wherein the first input data is the X-ray image dataset comprising an X-ray image and first metadata,

Applying a trained function to the input data, wherein output data is generated, wherein the output data comprises second metadata, wherein the first metadata and the second metadata are compared,
Providing the output data, wherein the first metadata are confirmed in case the first metadata and the second metadata are in agreement, or the first metadata are suggested to be corrected with the second metadata in case the first metadata and the second metadata are not in agreement.

The X-ray image dataset comprises the X-ray image which means a 2D- or 3D-image with pixel or voxel values.

According to an aspect of embodiments of the present invention, the first metadata and the second metadata comprise information regarding the body part and/or the view position.

According to an aspect of embodiments of the present invention, the X-ray image dataset is a DICOM image dataset.

According to an aspect of embodiments of the present invention, the second metadata is automatically corrected.

According to an aspect of embodiments of the present invention, a suggestion is displayed to the user to correct the first metadata with the second metadata and the user can confirm or decline the suggestion.

According to an aspect of embodiments of the present invention, a private DICOM tag is added in case the first metadata is corrected with the second metadata.

According to an aspect of embodiments of the present invention, the trained function determines the body part and/or the view position of the examination region in the X-ray image.

According to an aspect of embodiments of the present invention, the second metadata is available when the X-ray image is reviewed.

According to an aspect of embodiments of the present invention, the trained function is based on a convolutional neural network.

At least one embodiment of the present invention further relates to a computer-implemented method for providing a trained function, comprising:

Receiving input training data, wherein the input training data comprises an X-ray image dataset comprising an X-ray image and first metadata,
Receiving output training data, wherein the output training data is related to the input training data, wherein the output training data comprises second metadata,
Training a function based on the input training data and the output training data,
Providing the trained function.

During the training phase, the second metadata can correspond to the first metadata and/or the second metadata and the first metadata can be different. The second metadata can be determined by a specialist to create a groundtruth dataset which can be used as training data.

At least one embodiment of the present invention further relates to a checking system, comprising:
A first interface, configured for receiving first input data, wherein the first input data is an X-ray image dataset comprising an X-ray image and first metadata,
A computation unit, configured for applying a trained function to the input data, wherein output data is generated, wherein the output data comprises second metadata, wherein the first metadata and the second metadata are compared,
A second interface, configured for providing the output data, wherein the first metadata are confirmed in case the first metadata and the second metadata are in agreement, or the first metadata are suggested to be correct with the second metadata in case the first metadata and the second metadata are not in agreement.

At least one embodiment of the present invention further relates to a checking system comprising: a memory storing computer-executable instructions; and at least one processor configured to execute the computer-executable instructions to cause the checking system to perform the computer-implemented method for a post-acquisition check of an X-ray image dataset.

Embodiments of the present invention further relate to computer program product comprising instructions which, when the program is executed by a checking system, cause the checking system to carry out the method according to embodiments of the present invention.

Embodiments of the present invention further relate to a computer-readable medium comprising instructions which, when executed by a providing system, cause the providing system to carry out the method according to embodiments of the present invention

Embodiments of the present invention further relate to a training system, comprising:

A first training interface, configured for receiving input training data, wherein the input training data comprises an X-ray image dataset comprising an X-ray image and first metadata,
A second training interface, configured for receiving output training data, wherein the output training data is related to the input training data, wherein the output training data comprises second metadata,
A training computation unit, configured for training a function based on the input training data and the output training data,
A third training interface, configured for providing the trained function.

Embodiments of the present invention further relate to an X-ray system comprising the checking system according to embodiments of the present invention.

In general, parameters of a trained function can be adapted via training. In particular, supervised training, semi-supervised training, unsupervised training, reinforcement learning and/or active learning can be used. Furthermore, representation learning (an alternative term is “feature learning”) can be used. In particular, the parameters of the trained functions can be adapted iteratively by several steps of training.

In particular, a trained function can comprise a neural network, a support vector machine, a decision tree and/or a Bayesian network, and/or the trained function can be based on k-means clustering, Q-learning, genetic algorithms and/or association rules. In particular, a neural network can be a deep neural network, a convolutional neural network or a convolutional deep neural network. Furthermore, a neural network can be an adversarial network, a deep adversarial network and/or a generative adversarial network.

In the following, abbreviations can be used:

APAPC automatic post-acquisition protocol check
BP body part examined
BPVP body part & view position examined
CNN convolutional neural network
DICOM digital imaging and communications in medicine
PACS picture archiving and communication system
ML machine learning
VP view position

A standard imaging workflow usually comprises the following steps, preferably in the following order:

Image exam is requested
Operator selects imaging protocol and positions patient and system
Operator acquires X-ray image dataset
X-ray image dataset is processed on systemOperator reviews acquired X-ray image dataset
X-ray image dataset gets send to database (e.g. PACS)

In this workflow several errors can occur. Table 1 shows possible scenarios of mismatches between requested exam, selected protocols and examined body part.

Scenario #1 is a correct workflow. Scenarios #2 and #4 will usually be recognized at the time of image interpretation. Scenario #3 is often not recognized by the operator when reviewing the acquired image or by the radiologist interpreting the image.

This invention aims to address especially scenario #3 such that this scenario is recognized automatically after the image has been acquired and necessary correction steps can be performed.

TABLE 1
Examples of mismatches (scenarios #2, #3, #4)
between requested exam and selected
protocols and examined body part

| | requested | selected | examined |
|---|---|---|---|
| | exam | protocol | body |
| scenario | of . . . | for . . . | part |
| #1 | Hand | Hand | Hand |
| #2 | Hand | Hand | Foot |
| #3 | Hand | Foot | Hand |
| #4 | Hand | Foot | Foot |

The selected image acquisition protocol determines the value for the body part (BP) and view position (VP) that the imaging system writes into the DICOM header. Thus, an incorrectly selected protocol may lead to an incorrect BP and/or VP in the DICOM header.

This problem is addressed by incorporating the proposed method, which can be called APAPC (automatic post-acquisition protocol check), into the imaging exam workflow. The APAPC estimates BP and VP from the image content and compares it to the respective information defined in the selected clinical protocol. Mismatches are incorporated as feedback into the review process to enable corrections of potentially wrong DICOM header entries for BP and VP. In the clinical workflow, this could be achieved by directly informing the operator or radiologist, e.g., with a pop-up window or suggestion for correct BP and VP. An alternative workflow is to automatically correct the DICOM header and facilitate retrospective quality control (e.g., by marking corrected cases with a specific private DICOM tag).

After the X-ray image (dataset) has been processed on the system it is automatically sent to the APAPC component. This APAPC component can be installed:

locally on the imaging system or
centrally at one location in the hospital or
outside of the hospital (cloud environment).

When the image is received by the APAPC it contains the pixel data and meta information. Usually, medical images are stored in DICOM format. In the DICOM format information about the body part examined can be stored using DICOM tags such as Anatomic Region Sequence (0008,2218) or Body Part Examined (0018,0015). Information about the view position (e.g. AP, lateral, . . . ) can be stored using DICOM tags such as View Position (0018,5101).

The main steps of the AI system using the trained function are described in the following subsections. The description is made generally for any type of medical imaging system but as a specific example an x-ray imaging system can be imagined.

In a first step, the trained function classifies the body part from pixel data. The trained function uses the image's pixel data to classify the image content. A trained machine learning model such as a convolutional neural network (CNN; such as DenseNet takes the pixel data as input and assigned probability scores to each class it is trained on.

A simple example of training a neural network to classify between different body regions is to provide a number of body parts to the neural network as classification. Prerequisite is a labeled dataset of (X-ray) images and respective annotations (e.g., body parts derived from the DICOM header and verified by manual inspection). Based on this, a classifier neural network (e.g., based on DenseNet architecture) maps the medical input image to a pre-defined number of relevant body parts. Depending on the content of the input image, the respective entries of the output vector are set to zero (body part not existing in the image) or equal to one (body part existing in the image). This implies that during training the neural network transforms a medical image (X-ray image) to a vector with one-hot encoding (all but one entry equal to zero). This concept is very general and can also be applied to different output vector definitions (e.g., tuples of body part and viewing position) or neural network architectures.

A body part can be examined using various view positions (AP, lateral, and more). To predict body parts acquired from different views, first BPVP (body part & view position) classes are created (e.g. “chest-AP” and “chest-lateral”) on which the model is trained. Several BPVP classes can thus refer to one BP class.

The BPVP class with the highest probability score is denoted as BPVPpixel; that is, the BP and VP combination predicted from the pixel data. From the BPVPpixel combined class one can determine the individual classes BPpixel and VPpixel.

In a second step, the body part is extracted from meta information. The imaging system assigns BP and VP based on the selected protocol and stores this information in the DICOM header. To extract these classes (denoted as BPmeta and VPmeta) one can query the value of the respective DICOM tags.

In a third step, a check for internal consistency is performed. The two BP classes (BPpixel and BPmeta) are compared, and the two VP classes (VPpixel and VPmeta) are compared. If they are equal, it is assumed that the correct protocol has been selected and no further action is required. If they any of them are unequal, then a review workflow is initiated (see next step).

In a fourth step, a review workflow takes place. When a mismatch is detected, the operator receives a message to review the acquired image and to determine if the class from the meta data (BP and/or VP) data should be replaced by the class predicted from the pixel data (BP and/or VP). Thus, the operator receives a recommended new BP and/or VP class.

At the same time, the operator should be made aware that an incorrectly selected acquisition protocol could lead to non-optimal image quality and the image should be carefully reviewed for image quality. Alternatively, the correction could be made automatically when the system has only a low number of false positive alerts.

The ML model or the trained function can be trained on data outside of the institution where it is being used or from retrospective data from the institution where is being used or a combination of both. A continuous learning approach can be used to improve the trained function further.

In addition, the ML model or trained function can be continuously improved by adding new images to a pool of training images. After the image has been reviewed by the operator this image can be sent to a central location for this institution where the pool of training images is stored. In regular intervals a new training of the ML model is triggered. The (re-)trained model can then be used in all upcoming prediction tasks.

If the prediction runs locally on the system, the model must be copied several times to each local system (e.g. by a Wifi connection). To run the trained function or prediction locally can be especially useful for mobile X-ray systems. If the prediction runs in a central location, then it only needs to be copied once.

The proposed method provides automatic checks of every X-ray image acquired for internal consistency (pixel data and meta data refer to same body part and view position). This is fundamentally different to a manual check.

The automated check reduces time and effort compared to a manual check and can lead to higher detection of inconsistencies (as the check may be forgotten by the human operator). The protocol check is made directly after image acquisition and before the image is sent to PACS. Thus, the images generated by the medical imaging system are of higher quality (less incorrect DICOM header information expected). This can be a competitive advantage of this particular imaging system compared to other systems. The advantage of the classification approach (predict a specific BPVP class) is that the operator is given a specific recommendation to which new BP and/or VP class the DICOM header entry should be changed. By the continuous learning approach, the system is capable to adapt to specific types of examinations that occur at the particular institution.

### BRIEF DESCRIPTION OF THE DRAWINGS

Examples of embodiments of the present invention are explained in more detail below with regard to the drawings.

FIG. 1 a schematic representation of the method for a post-acquisition check of an X-ray image dataset in a first embodiment;

FIG. 2 a schematic representation of the method for a post-acquisition check of an X-ray image dataset in a second embodiment;

FIG. 3 a schematic representation of a neural network according to embodiments of the present invention; and

FIG. 4 a schematic representation of a convolutional neural network according to embodiments of the present invention; and

FIG. 5 a schematic representation of an X-ray imaging system.

### DETAILED DESCRIPTION

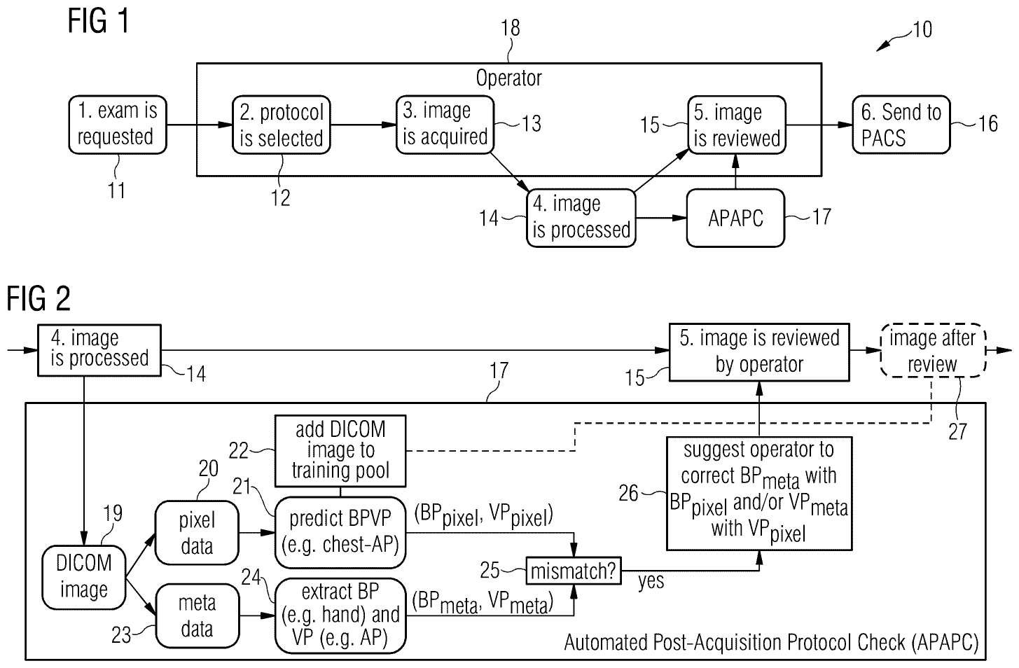

FIG. 1 displays a first embodiment of the method 10 for a post-acquisition check of an X-ray image dataset. In a first step 11, the exam is requested. In a second step 12, the protocol is selected by the operator. In a third step 13, the X-ray image is acquired. In a fourth step 14, the X-ray image is processed. In a fifth step 15, the X-ray image is reviewed. In a last step 16, the X-ray image is sent to PACS. After step 14 and before step 15, the trained function can be applied in step 17. The steps 12, 13, and 15 are performed in the operator domain 18.

FIG. 2 displays a second embodiment of the method for a post-acquisition check of an X-ray image dataset. After the image is processed in step 14, the DICOM image is received in step 19. Pixel data is extracted from the DICOM image in step 20. The trained function is applied to the pixel data in step 21 and a body part and view position are predicted. In step 23, the first meta data is extracted from the DICOM image. In step 24, the body part and the view position are extracted from the DICOM image. In step 25, the first metadata and the second metadata are compared. If there is a mismatch between the first metadata and the second metadata, second metadata as output are suggested and provided to the operator in step 26. Afterwards, the X-ray image is reviewed in step 15. In step 22, the DICOM image can be added to the training pool. In step 27, the X-ray image after review can be added to the training pool.

The computer-implemented method for a post-acquisition check of an X-ray image dataset, comprising the following steps:

Receiving input data 19, wherein the first input data is the X-ray image dataset comprising an X-ray image and first metadata,
Applying 17 a trained function to the input data, wherein output data is generated, wherein the output data comprises second metadata, wherein the first metadata and the second metadata are compared,
Providing 26 the output data, wherein the first metadata are confirmed in case the first metadata and the second metadata are in agreement, or the first metadata are suggested to be corrected with the second metadata in case the first metadata and the second metadata are not in agreement.

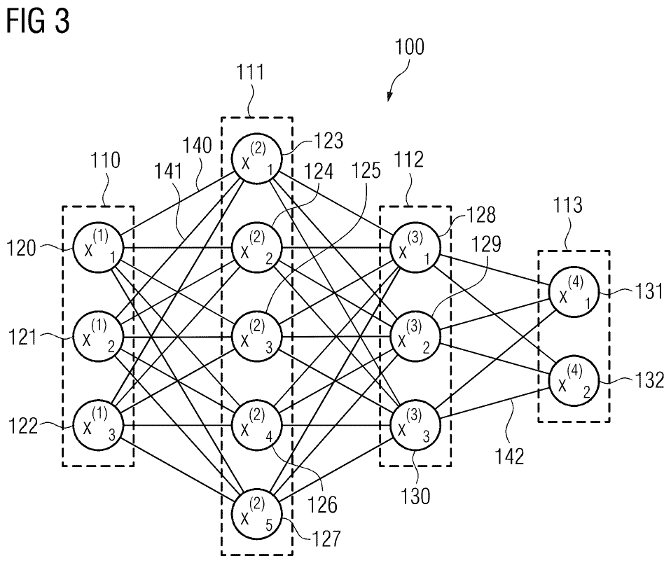

FIG. 3 displays an embodiment of an artificial neural network 100. Alternative terms for “artificial neural network” are “neural network”, “artificial neural net” or “neural net”.

The artificial neural network 100 comprises nodes 120, . . . , 132 and edges 140, . . . , 142, wherein each edge 140, . . . , 142 is a directed connection from a first node 120, . . . , 132 to a second node 120, . . . , 132. In general, the first node 120, . . . , 132 and the second node 120, . . . , 132 are different nodes 120, . . . , 132, it is also possible that the first node 120, . . . , 132 and the second node 120, . . . , 132 are identical. For example, in FIG. 1 the edge 140 is a directed connection from the node 120 to the node 123, and the edge 142 is a directed connection from the node 130 to the node 132. An edge 140, . . . , 142 from a first node 120, . . . , 132 to a second node 120, . . . , 132 is also denoted as “ingoing edge” for the second node 120, . . . , 132 and as “outgoing edge” for the first node 120, . . . , 132.

In this embodiment, the nodes 120, . . . , 132 of the artificial neural network 100 can be arranged in layers 110, . . . , 113, wherein the layers can comprise an intrinsic order introduced by the edges 140, . . . , 142 between the nodes 120, . . . , 132. In particular, edges 140, . . . , 142 can exist only between neighboring layers of nodes. In the displayed embodiment, there is an input layer 110 comprising only nodes 120, . . . , 122 without an incoming edge, an output layer 113 comprising only nodes 131, 132 without outgoing edges, and hidden layers 111, 112 in-between the input layer 110 and the output layer 113. In general, the number of hidden layers 111, 112 can be chosen arbitrarily. The number of nodes 120, . . . , 122 within the input layer 110 usually relates to the number of input values of the neural network, and the number of nodes 131, 132 within the output layer 113 usually relates to the number of output values of the neural network.

In particular, a (real) number can be assigned as a value to every node 120, . . . , 132 of the neural network 100. Here, x(n)i denotes the value of the i-th node 120, . . . , 132 of the n-th layer 110, . . . , 113. The values of the nodes 120, . . . , 122 of the input layer 110 are equivalent to the input values of the neural network 100, the values of the nodes 131, 132 of the output layer 113 are equivalent to the output value of the neural network 100. Furthermore, each edge 140, . . . , 142 can comprise a weight being a real number, in particular, the weight is a real number within the interval [−1, 1] or within the interval [0, 1]. Here, w(m,n)i,j denotes the weight of the edge between the i-th node 120, . . . , 132 of the m-th layer 110, . . . , 113 and the j-th node 120, . . . , 132 of the n-th layer 110, . . . , 113. Furthermore, the abbreviation w(n)i,j is defined for the weight w(n,n+1)i,j.

In particular, to calculate the output values of the neural network 100, the input values are propagated through the neural network. In particular, the values of the nodes 120, . . . , 132 of the (n+1)-th layer 110, . . . , 113 can be calculated based on the values of the nodes 120, . . . , 132 of the n-th layer 110, . . . , 113 by

xⱼ⁽ⁿ⁺¹⁾=f(Σᵢxᵢ⁽ⁿ⁾·w_{i,j}⁽ⁿ⁾).

Herein, the function f is a transfer function (another term is “activation function”). Known transfer functions are step functions, sigmoid function (e.g. the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 110 are given by the input of the neural network 100, wherein values of the first hidden layer 111 can be calculated based on the values of the input layer 110 of the neural network, wherein values of the second hidden layer 112 can be calculated based in the values of the first hidden layer 111, etc.

In order to set the values w(m,n)i,j for the edges, the neural network 100 has to be trained using training data. In particular, training data comprises training input data and training output data (denoted as ti). For a training step, the neural network 100 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 100 (backpropagation algorithm). In particular, the weights are changed according to

w′_{i,j}⁽ⁿ⁾=w_{i,j}⁽ⁿ⁾−γ·δⱼ⁽ⁿ⁾·xᵢ⁽ⁿ⁾

δⱼ⁽ⁿ⁾=(Σₖδₖ⁽ⁿ⁺¹⁾·w_{j,k}⁽ⁿ⁺¹⁾)·f′(Σᵢ⁽ⁿ⁾·w_{i,j}⁽ⁿ⁾)δⱼ⁽ⁿ⁾=(xₖ⁽ⁿ⁺¹⁾−tⱼ⁽ⁿ⁺¹⁾)·f′(Σᵢxᵢ⁽ⁿ⁾·w_{i,j}⁽ⁿ⁾)

wherein γ is a learning rate, and the numbers δ(n)j can be recursively calculated as based on δ(n+1)j, if the (n+1)-th layer is not the output layer, and
if the (n+1)-th layer is the output layer 113, wherein f′ is the first derivative of the activation function, and y(n+1)j is the comparison training value for the j-th node of the output layer 113.

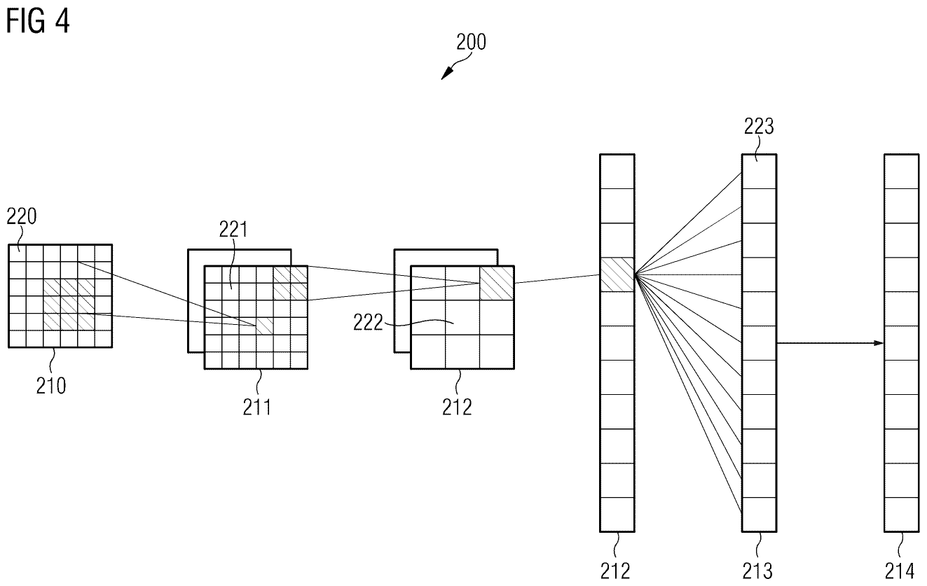

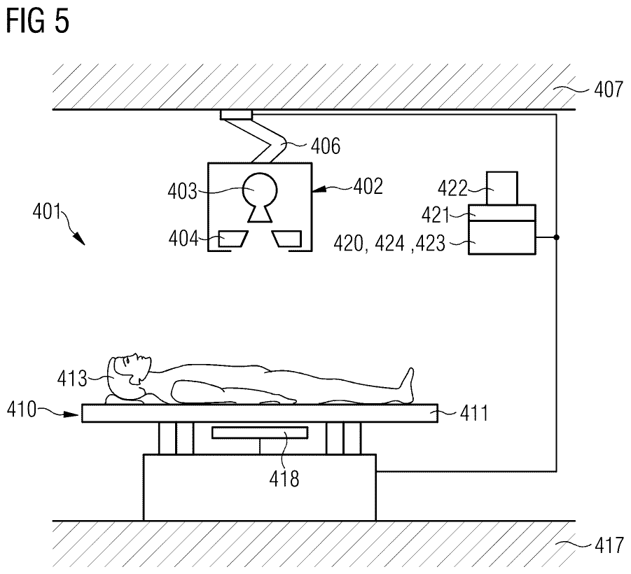

FIG. 4 displays an embodiment of a convolutional neural network 200. In the displayed embodiment, the convolutional neural network comprises 200 an input layer 210, a convolutional layer 211, a pooling layer 212, a fully connected layer 213 and an output layer 214. Alternatively, the convolutional neural network 200 can comprise several convolutional layers 211, several pooling layers 212 and several fully connected layers 213, as well as other types of layers. The order of the layers can be chosen arbitrarily, usually fully connected layers 213 are used as the last layers before the output layer 214.

In particular, within a convolutional neural network 200 the nodes 220, . . . , 224 of one layer 210, . . . , 214 can be considered to be arranged as a d-dimensional matrix or as a d-dimensional image. In particular, in the two-dimensional case the value of the node 220, . . . , 224 indexed with i and j in the n-th layer 210, . . . , 214 can be denoted as x(n)[i,j]. However, the arrangement of the nodes 220, . . . , 224 of one layer 210, . . . , 214 does not have an effect on the calculations executed within the convolutional neural network 200 as such, since these are given solely by the structure and the weights of the edges.

In particular, a convolutional layer 211 is characterized by the structure and the weights of the incoming edges forming a convolution operation based on a certain number of kernels. In particular, the structure and the weights of the incoming edges are chosen such that the values x(n)k of the nodes 221 of the convolutional layer 211 are calculated as a convolution x(n)k=Kk*x(n−1) based on the values x(n−1) of the nodes 220 of the preceding layer 210, where the convolution * is defined in the two-dimensional case as

xₖ⁽ⁿ⁾[i,j]=(Kₖ*x⁽ⁿ⁻¹⁾)[i,j]=Σ_{i′}Σ_{j′}Kₖ[i′,j′]·x⁽ⁿ⁻¹⁾[i−i′, j−j′].

Here the k-th kernel Kk is a d-dimensional matrix (in this embodiment a two-dimensional matrix), which is usually small compared to the number of nodes 220, . . . , 224 (e.g. a 3×3 matrix, or a 5×5 matrix). In particular, this implies that the weights of the incoming edges are not independent, but chosen such that they produce said convolution equation. In particular, for a kernel being a 3×3 matrix, there are only 9 independent weights (each entry of the kernel matrix corresponding to one independent weight), irrespectively of the number of nodes 220, . . . , 224 in the respective layer 210, . . . , 214. In particular, for a convolutional layer 211 the number of nodes 221 in the convolutional layer is equivalent to the number of nodes 220 in the preceding layer 210 multiplied with the number of kernels.

If the nodes 220 of the preceding layer 210 are arranged as a d-dimensional matrix, using a plurality of kernels can be interpreted as adding a further dimension (denoted as “depth” dimension), so that the nodes 221 of the convolutional layer 221 are arranged as a (d+1)-dimensional matrix. If the nodes 220 of the preceding layer 210 are already arranged as a (d+1)-dimensional matrix comprising a depth dimension, using a plurality of kernels can be interpreted as expanding along the depth dimension, so that the nodes 221 of the convolutional layer 221 are arranged also as a (d+1)-dimensional matrix, wherein the size of the (d+1)-dimensional matrix with respect to the depth dimension is by a factor of the number of kernels larger than in the preceding layer 210.

The advantage of using convolutional layers 211 is that spatially local correlation of the input data can exploited by enforcing a local connectivity pattern between nodes of adjacent layers, in particular by each node being connected to only a small region of the nodes of the preceding layer.

In the displayed embodiment, the input layer 210 comprises 36 nodes 220, arranged as a two-dimensional 6×6 matrix. The convolutional layer 211 comprises 72 nodes 221, arranged as two two-dimensional 6×6 matrices, each of the two matrices being the result of a convolution of the values of the input layer with a kernel. Equivalently, the nodes 221 of the convolutional layer 211 can be interpreted as arranges as a three-dimensional 6×6×2 matrix, wherein the last dimension is the depth dimension.

A pooling layer 212 can be characterized by the structure and the weights of the incoming edges and the activation function of its nodes 222 forming a pooling operation based on a non-linear pooling function f. For example, in the two dimensional case the values x(n) of the nodes 222 of the pooling layer 212 can be calculated based on the values x(n−1) of the nodes 221 of the preceding layer 211 as

x⁽ⁿ⁾[i,j]=f(x⁽ⁿ⁻¹⁾[id₁, jd₂], . . . , x⁽ⁿ⁻¹⁾[id₁+d₁−1, jd₂+d₂−1])

In other words, by using a pooling layer 212 the number of nodes 221, 222 can be reduced, by replacing a number d1·d2 of neighboring nodes 221 in the preceding layer 211 with a single node 222 being calculated as a function of the values of said number of neighboring nodes in the pooling layer. In particular, the pooling function f can be the max-function, the average or the L2-Norm. In particular, for a pooling layer 212 the weights of the incoming edges are fixed and are not modified by training.

The advantage of using a pooling layer 212 is that the number of nodes 221, 222 and the number of parameters is reduced. This leads to the amount of computation in the network being reduced and to a control of overfitting.

In the displayed embodiment, the pooling layer 212 is a max-pooling, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

A fully-connected layer 213 can be characterized by the fact that a majority, in particular, all edges between nodes 222 of the previous layer 212 and the nodes 223 of the fully-connected layer 213 are present, and wherein the weight of each of the edges can be adjusted individually.

In this embodiment, the nodes 222 of the preceding layer 212 of the fully-connected layer 213 are displayed both as two-dimensional matrices, and additionally as non-related nodes (indicated as a line of nodes, wherein the number of nodes was reduced for a better presentability). In this embodiment, the number of nodes 223 in the fully connected layer 213 is equal to the number of nodes 222 in the preceding layer 212. Alternatively, the number of nodes 222, 223 can differ.

Furthermore, in this embodiment the values of the nodes 224 of the output layer 214 are determined by applying the Softmax function onto the values of the nodes 223 of the preceding layer 213. By applying the Softmax function, the sum of the values of all nodes 224 of the output layer is 1, and all values of all nodes 224 of the output layer are real numbers between 0 and 1. In particular, if using the convolutional neural network 200 for categorizing input data, the values of the output layer can be interpreted as the probability of the input data falling into one of the different categories.

A convolutional neural network 200 can also comprise a ReLU (acronym for “rectified linear units”) layer. In particular, the number of nodes and the structure of the nodes contained in a ReLU layer is equivalent to the number of nodes and the structure of the nodes contained in the preceding layer. In particular, the value of each node in the ReLU layer is calculated by applying a rectifying function to the value of the corresponding node of the preceding layer. Examples for rectifying functions are f(x)=max(0,x), the tangent hyperbolics function or the sigmoid function.

In particular, convolutional neural networks 200 can be trained based on the backpropagation algorithm. For preventing overfitting, methods of regularization can be used, e.g. dropout of nodes 220, . . . , 224, stochastic pooling, use of artificial data, weight decay based on the L1 or the L2 norm, or max norm constraints.

FIG. 5 shows an exemplary embodiment of an X-ray (imaging) system 401, especially a radiography system, according to the present invention. The X-ray system 401 has a patient positioning device 410 with a table 411 fixed to the floor 417. The object 413 lies on the table 411. The patient positioning device 410 further comprises an X-ray detector unit 418.

The X-ray system 401 comprises an X-ray source 403 and an X-ray detector unit 418. The X-ray source unit 402, which comprises the X-ray source 403 and a collimator unit 404. The X-ray source unit 402 can be connected to the ceiling 407 of the examination room via a ceiling mount 406. Via the ceiling mount 406, the X-ray source 403 can be moved.

The X-ray system 401 may also comprise an input unit 421 and an output unit 422. The input unit 421 and the output unit 422 may be connected to the control unit 420. The control unit 420 may comprise the checking system. The control unit 420 may further comprise or be connected to the training system 424.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been further illustrated in detail by the preferred embodiments, the present invention is not limited by the disclosed examples and other variations may be derived therefrom by those skilled in the art without departing from the scope of protection of the present invention.

What is claimed is:

1. A computer-implemented method for a post-acquisition check of an X-ray image dataset, the computer-implemented method comprising:
receiving input data, wherein the input data includes the X-ray image dataset, which includes an X-ray image and first metadata;
applying a trained function to the input data to generate output data, wherein the output data includes second metadata, and wherein the first metadata and the second metadata are compared; and
providing the output data, wherein the first metadata are confirmed in case the first metadata and the second metadata agree, or the first metadata are suggested to be corrected with the second metadata in case the first metadata and the second metadata do not agree.

2. The computer-implemented method according to claim 1, wherein the first metadata and the second metadata comprise information regarding at least one of a body part or a view position of an examination region in the X-ray image.

3. The computer-implemented method according to claim 1, wherein the X-ray image dataset is a DICOM image dataset.

4. The computer-implemented method according to claim 1, wherein the second metadata is automatically corrected.

5. The computer-implemented method according to claim 1, wherein a suggestion to correct the first metadata with the second metadata is displayed to a user for confirming or declining the suggestion.

6. The computer-implemented method according to claim 1, wherein a private DICOM tag is added in case the first metadata is corrected using the second metadata.

7. The computer-implemented method according to claim 1, wherein the trained function determines at least one of a body part or a view position of an examination region in the X-ray image.

8. The computer-implemented method according to claim 1, wherein the second metadata is available when the X-ray image is reviewed.

9. The computer-implemented method according to claim 1, wherein the trained function is based on a convolutional neural network.

10. A computer-implemented method for providing a trained function, the computer-implemented method comprising:
receiving input training data, wherein the input training data includes an X-ray image dataset, which includes an X-ray image and first metadata;
receiving output training data, wherein the output training data is related to the input training data, and wherein the output training data includes second metadata,
training a function based on the input training data and the output training data to obtain the trained function; and
providing the trained function.

11. A checking system, comprising:
a first interface configured to receive input data, wherein the input data includes an X-ray image dataset, which includes an X-ray image and first metadata;
a computation unit configured to apply a trained function to the input data to generate output data, wherein the output data includes second metadata, and wherein the first metadata and the second metadata are compared; and
a second interface configured to provide the output data, wherein the first metadata are confirmed in case the first metadata and the second metadata agree, or the first metadata are suggested to be corrected with the second metadata in case the first metadata and the second metadata do not agree.

12. A non-transitory computer-readable medium storing instructions which, when executed by a checking system, cause the checking system to perform the method of claim 1.

13. A non-transitory computer-readable medium storing instructions which, when executed by a providing system, cause the providing system to perform the method of claim 10.

14. A training system, comprising:
a first training interface configured to receive input training data, wherein the input training data includes an X-ray image dataset, which includes an X-ray image and first metadata;
a second training interface configured to receive output training data, wherein the output training data is related to the input training data, and wherein the output training data includes second metadata;
a training computation unit configured to train a function based on the input training data and the output training data to obtain a trained function; and
a third training interface configured to provide the trained function.

15. An X-ray system comprising the checking system according to claim 11.

16. The computer-implemented method according to claim 2, wherein a suggestion to correct the first metadata with the second metadata is displayed to a user for confirming or declining the suggestion.

17. The computer-implemented method according to claim 16, wherein a private DICOM tag is added in case the first metadata is corrected using the second metadata.

18. The computer-implemented method according to claim 17, wherein the second metadata is available when the X-ray image is reviewed.

19. The computer-implemented method according to claim 17, wherein the trained function is based on a convolutional neural network.

20. A checking system comprising:
a memory storing computer-executable instructions; and
at least one processor configured to execute the computer-executable instructions to cause the checking system to perform the method of claim 1.
